(19) United States
(12) Patent Application Publication
(10) Pub. No.: US 2006/0224074 A1
(43) Pub. Date: Oct. 5, 2006

(54) Heartbeat measuring apparatus

(75) Inventors: Kazushige Ouchi, Kanagawa (JP); Takuji Suzuki, Kanagawa (JP); Akihisa Moriya, Kanagawa (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: KABUSHIKI KAISHA TOSHIBA, Minato-ku (JP)

(21) Appl. No.: 11/366,535

(22) Filed: Mar. 3, 2006

(30) Foreign Application Priority Data
Mar. 29, 2005 (JP) 2005-096354

(51) Int. Cl. A61B 5/0402

(52) U.S. Cl. 600/513

(57) ABSTRACT

A heartbeat measuring apparatus includes an biological information acquiring unit that acquires biological information derived from heartbeats, pulses or the like of a user; a body-movement detecting unit that detects a body movement of the user; a reference information generating unit that generates reference information, which serves as a reference indicating the heartbeats or pulses for comparison, based upon the biological information acquired by the biological information acquiring unit after completion of a detection of the body movement; a waveform similarity calculating unit that calculates a waveform similarity between the reference information and biological information acquired after the reference information is generated; an extreme value acquiring unit that specifies an extreme value of the heartbeats or pulses from a plurality of waveform similarities, and acquires extreme value time at which the extreme value is obtained; and an biological information analyzing unit that analyzes biological information of the user based upon a time interval between the extreme value times, to obtain desired information.

### CROSS-REFERENCE TO RELATED APPLICATIONS

This application is based upon and claims the benefit of priority from the prior Japanese Patent Application No. 2005-096354, filed on Mar. 29, 2005; the entire contents of which are incorporated herein by reference.

### BACKGROUND OF THE INVENTION

1. Field of the Invention

This invention relates to a heartbeat measuring apparatus and a heartbeat measuring method, and more specifically concerns a technique for obtaining measurements of biological information such as heartbeats and pulse waves from a body of a user so as to calculate the heartbeat and analyze autonomic nerve.

2. Description of the Related Art

One conventional method generally employed for measuring a sleeping state is an all-night polygraph inspection, during which a plurality of sensors are attached to a patient's body for the measurements of brain waves, heartbeats, electromyogram, respiration, blood oxygen saturation levels (SpO2), and the like for analysis of a sleeping state. Implementation of such inspection lasts for two nights and three days in a specialized facility such as a hospital. Hence, the inspection is disadvantageous in that the patient has to put up with unpleasant feelings caused by the attached sensors, the long time required for the inspection, and costs of the inspection, and that the doctor also needs to prepare for the place used for the inspection and conduct time-consuming tasks for the inspection.

On the other hand, an attempt has been made to realize a simplified estimation of a sleeping state with a use of a mat-type sensor and the like. For example, a technique described in “Unconstrained and Noninvasive Automatic Measurement of Respiration and Heart Rates Using a Strain Gauge” Vol. 36, No. 3, p227-233 in collected papers of the Society of Instrument and Control Engineers, written by Shogo Tanaka, realizes measurements of pressure changes caused by breathing, heartbeats, and body movements with a pressure sensor or the like for utilization in the estimation of the sleeping state. According to other techniques, the estimation of the sleeping state is realized based on the variation in the heart rates, or based on the activities of the autonomic nerve system known from the variation in the heart rates.

In addition, some proposals are made to enhance the accuracy of the technique for estimating the sleeping state based upon data acquired by the pressure sensor. For example, Japanese Patent Publication No. 2004-89314 (hereinafter, referred to as Document 1) discloses an invention for estimating the variation of heart rates based on the pressure changes measured by the pressure sensor, in which, in order to estimate variations in heartbeats based upon pressure fluctuations acquired by a pressure sensor or the like, template data, derived from electrocardiogram waveform data based upon a predetermined heartbeat signal, is stored and the stored template data is compared with data acquired by the pressure sensor so that the heart rate is calculated.

According to the invention disclosed in Document 1, however, when the heartbeat is measured by the sensor, the characteristic (shape, amplitude, or the like) of the waveform to be measured changes according to the postures of the user. Hence, the use of the same template data ends up in impossibility of uniform determination of the heartbeat detection method, or in impossibility of stable all-night measurement due to deviations in the measurement accuracy caused by the changing postures of the user, if the heartbeat detection method is determined fixedly.

### SUMMARY OF THE INVENTION

According to one aspect of the present invention, a heartbeat measuring apparatus includes an biological information acquiring unit that acquires biological information derived from heartbeats, pulses or the like of a user; a body-movement detecting unit that detects a body movement of the user; a reference information generating unit that generates reference waveform information, which serves as a reference indicating the heartbeats or pulses for comparison, based upon the biological information acquired by the biological information acquiring unit after completion of a detection of the body movement; a waveform similarity calculating unit that calculates a waveform similarity between the reference waveform information and biological information acquired after the reference waveform information is generated; an extreme value acquiring unit that specifies an extreme value of the heartbeats or pulses from a plurality of waveform similarities, and acquires extreme value time at which the extreme value is obtained; and an biological information analyzing unit that analyzes biological information of the user based upon a time interval between the extreme value times, to obtain desired information.

According to another aspect of the present invention, a method of measuring a heartbeat includes acquiring biological information derived from heartbeats, pulses or the like of a user; detecting a body movement of the user; generating reference waveform information, which serves as a reference indicating the heartbeats or pulses for comparison, based upon the acquired biological information after completion of a detection of the body movement; calculating a waveform similarity between the reference waveform information and biological information acquired after the reference waveform information is generated; acquiring an extreme value time at which an extreme value is obtained by specifying the extreme value of the heartbeats or pulses from a plurality of waveform similarities; and acquiring desired information on a living body of the user by analyzing the biological information of the user based upon a time interval between the extreme value times.

### BRIEF DESCRIPTION OF THE DRAWINGS

FIG. 1 is a block diagram that shows a structure of a heartbeat measuring apparatus in accordance with a first embodiment of the present invention;

FIG. 2 shows a sensor unit that is connected to the heartbeat measuring apparatus of the first embodiment, that is, a mat-type sensor, which is made in contact with the body of the user, and detects biological information concerning the heartbeat or the pulse;

FIG. 3 shows a sensor unit connected to a heartbeat measuring apparatus in accordance with an embodiment different from the first embodiment, that is, a pillow-type sensor unit, which detects biological information;

FIG. 4 shows a sensor unit connected to a heartbeat measuring apparatus in accordance with an embodiment different from the first embodiment, that is, a sensor unit of a photoelectric pulse-wave measuring system, which detects biological information;

FIG. 5 is a chart of one example of amplified waveform data obtained by amplification of biological information, detected by the sensor unit of the heartbeat measuring apparatus of the first embodiment, by a biological amplifying unit;

FIG. 6 is a chart of one example of waveform data of a frequency band indicating breathing that is included in the waveform data amplified by the biological amplifying unit of the heartbeat measuring apparatus of the first embodiment;

FIG. 7 is a chart of one example of waveform data of a frequency band indicating heartbeat that is included in the waveform data amplified by the biological amplifying unit of the heartbeat measuring apparatus of the first embodiment;

FIG. 8 is a chart of one example of waveform data of a frequency band indicating the heartbeat in accordance with biological information acquired by an biological information acquiring unit upon occurrence of body movements;

FIG. 9 is an explanatory chart of an operation in which, by using, as a template, waveform data of heartbeat corresponding to one beat that has been cut off after the completion of the body movements in waveform data in the frequency band indicating the heartbeat in accordance with the heartbeat measuring apparatus of the first embodiment, the succeeding waveform data is evaluated on the similarity relationship;

FIG. 10 is an explanatory chart that shows an example in which based upon a peak value detected by a heartbeat peak value detecting unit, a template generating unit of the heartbeat measuring apparatus of the first embodiment cuts off one beat portion from waveform data of a frequency area indicating the heartbeat;

FIG. 11 is an explanatory chart that shows a sequence of processes in which a waveform similarity calculating unit of the heartbeat measuring apparatus of the first embodiment calculates a correlation coefficient as the waveform similarity by using the template;

FIG. 12A is a chart of a change in the correlation coefficient when the template is evaluated as an appropriate one by an evaluating unit of the heartbeat measuring apparatus of the first embodiment;

FIG. 12B is a chart of a change in the correlation coefficient when the template is evaluated as an inappropriate one by the evaluating unit of the heartbeat measuring apparatus of the first embodiment;

FIG. 13 is a chart of one example of a correlation coefficient calculated by the waveform similarity calculating unit of the heartbeat measuring apparatus of the first embodiment, with a value forming a peak value detected by a peak value detecting unit being enclosed by a circle;

FIG. 14 shows an example of a screen displayed on a monitor, which includes the results of processes carried out by a display processing unit of the heartbeat measuring apparatus of the first embodiment;

FIG. 15 is a flow chart that shows a sequence of processes from calculating heart rate and LF, HF values and the like based upon biological information detected by the sensor unit up to displaying the results on the monitor in the heartbeat measuring apparatus in accordance with the first embodiment;

FIG. 16 is a block diagram that shows a structure of a heartbeat measuring apparatus in accordance with a second embodiment;

FIG. 17 shows a body-movement sensor unit connected to a heartbeat measuring apparatus in accordance with the second embodiment, and exemplifies a case in which the sensor unit is attached to the user;

FIG. 18 is a chart of one example of a signal that is outputted when a 3-axis accelerometer is used as the body-movement sensor unit connected to the heartbeat measuring apparatus of the second embodiment;

FIG. 19 is an explanatory chart of an example in which based upon a peak value detected by a heartbeat peak value detecting unit, a template generating unit of a heartbeat measuring apparatus in accordance with a first modification cuts off one beat portion from waveform data of a frequency area indicating the heartbeat;

FIG. 20 is an explanatory drawing that indicates a % sequence of processes in which a template generating unit in accordance with the heartbeat measuring apparatus of a second modification calculates waveform data that is used as a template from a plurality of acquired waveform data corresponding to one beat portion of the heartbeat;

FIG. 21 is an explanatory drawing that indicates a sequence of processes in which a template generating unit in accordance with the heartbeat measuring apparatus of a third modification acquires waveform data that is used as a template from a plurality of acquired waveform data corresponding to one beat portion of the heartbeat;

FIG. 22 is an explanatory drawing that indicates a sequence of processes in which a template generating unit in accordance with the heartbeat measuring apparatus of the third modification selects waveform data that is used as a template from a plurality of acquired waveform data corresponding to one beat portion of the heartbeat;

FIG. 23A is a chart of a change in the correlation coefficient when the template is evaluated as an appropriate one by the evaluating unit of a heartbeat measuring apparatus in accordance with a fourth modification; and

FIG. 23B is a chart of a change in the correlation coefficient when the template is evaluated as an inappropriate one by the evaluating unit of a heartbeat measuring apparatus of the fourth modification.

### DETAILED DESCRIPTION OF THE INVENTION

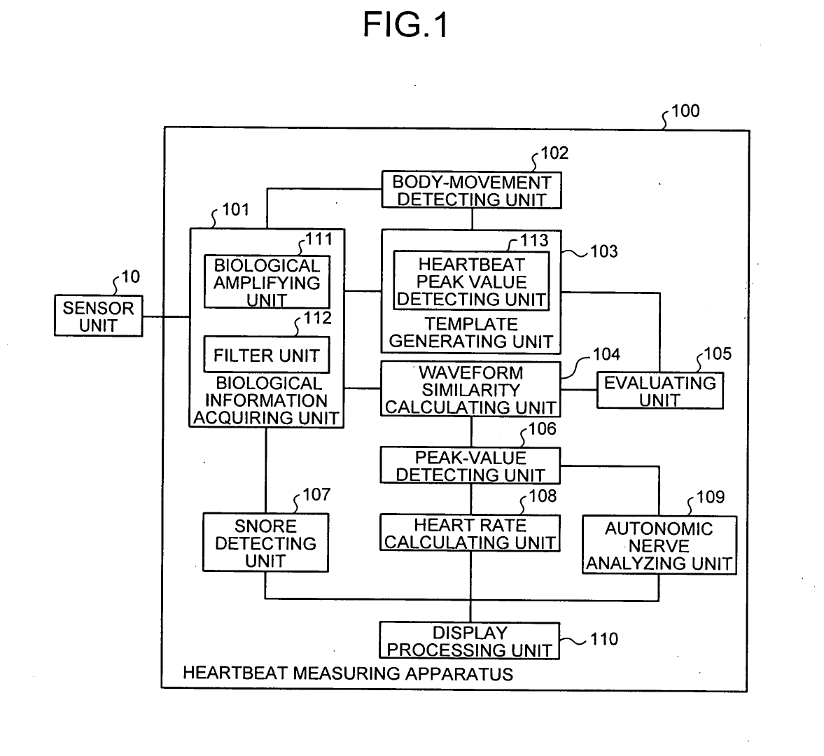

FIG. 1 is a block diagram that shows a structure of a heartbeat measuring apparatus 100 in accordance with a first embodiment of the present invention. As shown in this figure, the heartbeat measuring apparatus includes an biological information acquiring unit 101, a body-movement detecting unit 102, a template generating unit 103, a waveform similarity calculating unit 104, an evaluating unit 105, a peak-value detecting unit 106, a snore detecting unit 107, a heart rate calculating unit 108, an autonomic nerve analyzing unit 109, and a display processing unit 110. With this structure, the heartbeat measuring apparatus 100 allows for measurements of the heart rate, the autonomic nerve analysis or the like, by utilizing a template that is formed based upon waveform data detected from the user by the sensor unit 10. Moreover, after a body movement of the user such as turning over in bed, the heartbeat measuring apparatus 100 forms a template that is appropriate for the state after the body movement, and by using this template, the heart rate is calculated or the autonomic nerve is analyzed so that the accuracy of the apparatus is improved.

The template, which corresponds to reference waveform information of the present invention, is waveform data generated based upon biological information that is derived from pulses or heartbeats and acquired by the biological information acquiring unit 101, and is used as a reference with which waveform data, detected by the sensor, is compared.

The biological information acquiring unit 101, which is provided with a biological amplifying unit 111 and a filter unit 112, acquires biological information derived from the frequency band of heartbeat based upon living body signals including the heartbeat, pulse and the like, which are inputted from the sensor unit 10 that is made in contact with the body of the user. The sensor unit 10, connected to the biological information acquiring unit 101, may be prepared as any sensor as long as it allows detection of living body signals relating to the heartbeat and the like from the user's body, and, for example, a mat-type sensor is used in the present embodiment.

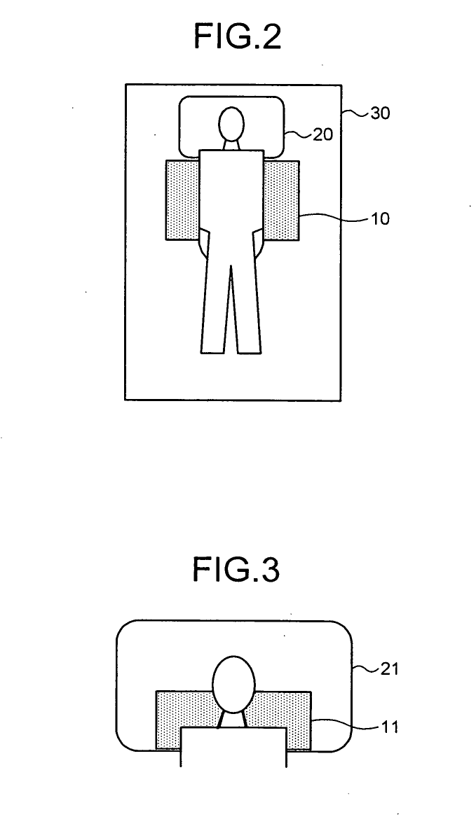

FIG. 2 shows the sensor unit 10 that is connected to the heartbeat measuring apparatus 100 of the present embodiment, and is prepared as a mat-type sensor that is made in contact with the user's body, and used for detecting biological information including the heartbeat and pulse. In the mat-type sensor unit 10 of FIG. 2, an air mat is filled with air, and a pressure change inside the air mat caused in response to an action of a biological object (beats of the heart, breathing, body movements and the like) is detected so that the action of the biological object is confirmed. Here, the mat-type sensor is not intended to be limited to the air type, and, for example, that of a strain gauge type or that utilizes a light diffusion change due to pressure may be used.

Moreover, with respect to a sensor to be connected to the biological information acquiring unit 101, which is different from that of the present embodiment, for example, a pillow type sensor or a sensor unit of a photoelectric pulse-wave measuring system may be adopted.

FIG. 3 shows a sensor unit connected to a heartbeat measuring apparatus relating to an embodiment different from the present embodiment, that is, a pillow-type sensor unit 11 that detects biological information. In the same manner as the mat-type sensor unit 10, the pillow-type sensor unit 11, shown in the present figure, is used for capturing a pressure change in response to a biological action of the user. Here, sensors, which are installed inside the pillow-type sensor 11 and located on the lower side or the upper side of the pillow, are mainly positioned along the neck to the shoulder of the user so that the biological action of the user is measured.

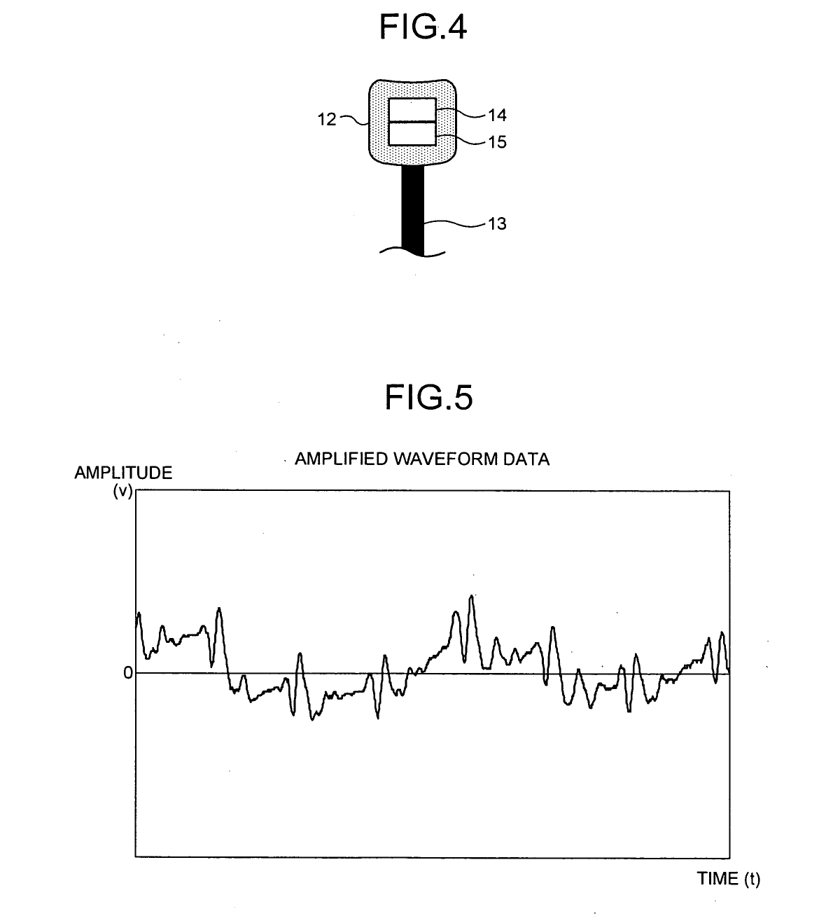

FIG. 4 shows a sensor unit connected to a heartbeat measuring apparatus relating to an embodiment different from the present embodiment, that is, a sensor unit 12 of a photoelectric pulse-wave measuring system, which detects biological information. The sensor unit 12 shown in the present figure is attached to a finger of the user, and near infrared rays or red or blue light rays are applied to the skin from a light-emitting element 14 so that a change in the intensity of reflected light or transmitted light corresponding to a blood current change due to the absorption of light rays by the hemoglobin is received by a light-receiving element 15 to detect biological information including the pulse wave. As described earlier, any sensor may be used as long as it can detect biological information including the pulse wave, and with respect to another example, a pressure pulse wave measuring system, which receives beats in the vicinity of an artery as a pressure, may be applied to the sensor unit.

The biological amplifying unit 111 amplifies the biological information detected by the sensor unit 10. More specifically, a voltage value indicating biological information, detected by the sensor unit 10, is amplified by the biological amplifying unit 111. Here, any method may be used as the method by which the biological amplifying unit 111 amplifies the voltage value. By allowing the biological amplifying unit 111 to amplify the voltage value, biological information obtained by the sensor unit 10 can be detected, even when it is very weak.

FIG. 5 shows one example of waveform data that is obtained through amplification of biological information detected by the sensor unit 10 with the use of the biological amplifying unit 111. As shown in this figure, the amplitudes of biological information that includes the heartbeat, pulse, breathing, and body movement of the user, and is amplified by the biological amplifying unit 111 fluctuate over time. Since the biological information detected by the mat-type sensor unit 10 of the present embodiment also includes measured waveform data other than heartbeats (for example, waveform data of breathing), a filter unit 112 removes these waveform data. More specifically, the filter unit 112, which is prepared as a band pass filter that removes frequency bands other than the frequency band indicating the heartbeats, makes it possible to remove breathing waveform data corresponding to a frequency lower than that of the heartbeat waveform and snoring waveform data corresponding to a frequency component higher than that of the heartbeat waveform. For example, the filter unit 112 eliminates the breathing waveform data by removing frequencies of 0.5 Hz or less, and also eliminates the snoring waveform data by removing frequencies of 5 Hz or more. Here, the band pass filter used as the filter unit 112 does not limit the value of frequency bands to be removed to the above-mentioned values, and any band pass filter may be used as long as it removes frequency bands other than the frequency band by which the heartbeat waveform can be measured. In the present embodiment, the band pass filter is used as the filter unit 112; however, a high pass filter may be used in order to eliminate only low-frequency band components such as breathing waveform. The following description will describe specific waveform data.

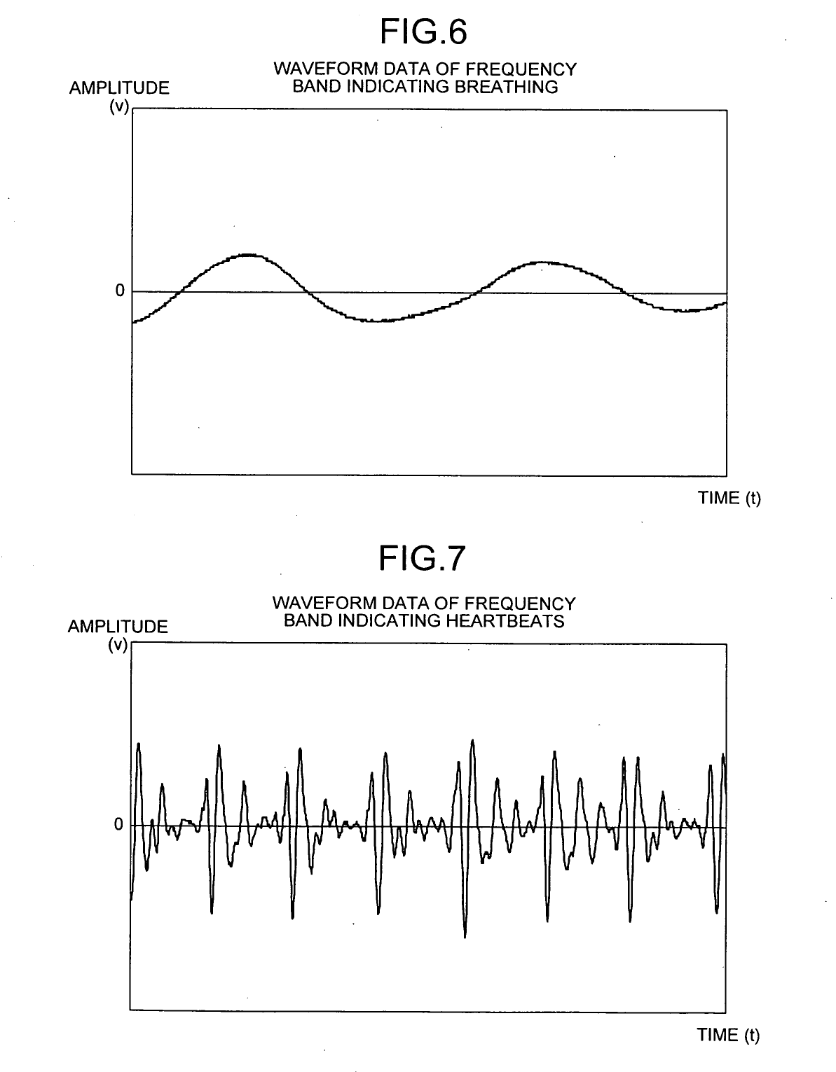

FIG. 6 shows one example of waveform data of a frequency band indicating breathing that is included in the waveform data amplified by the biological amplifying unit 111. FIG. 7 shows one example of waveform data of a frequency band indicating heartbeats, which is included in the waveform data amplified by the biological amplifying unit 111. The waveform data shown in FIG. 5, which has been amplified by the biological amplifying unit 111, has been measured as multiplexed waveform data including waveform data of a frequency band indicating breathing as shown in FIG. 6, waveform data of a frequency band indicating heartbeats as shown in FIG. 7 and waveform data of a high-frequency band indicating snoring. Therefore, by eliminating the waveform data of the frequency band indicating breathing as shown in FIG. 6 and waveform data of the high-frequency band indicating snoring by using the band pass filter of the filter unit 112 from the waveform data amplified by the biological amplifying unit 111 shown in FIG. 5, it becomes possible to obtain waveform data of the frequency band indicating heartbeats as shown in FIG. 7.

Thus, by eliminating waveform data of excessive frequency bands by the use of the filter unit 112, it becomes possible to easily acquire the waveform data of frequency band indicating heartbeats.

Based upon the waveform data of heartbeats which has been obtained by eliminating excessive waveform data from the data obtained by the biological information acquiring unit 101 through the filter unit 112, the body-movement detecting unit 102 detects whether or not any body movement is occurring.

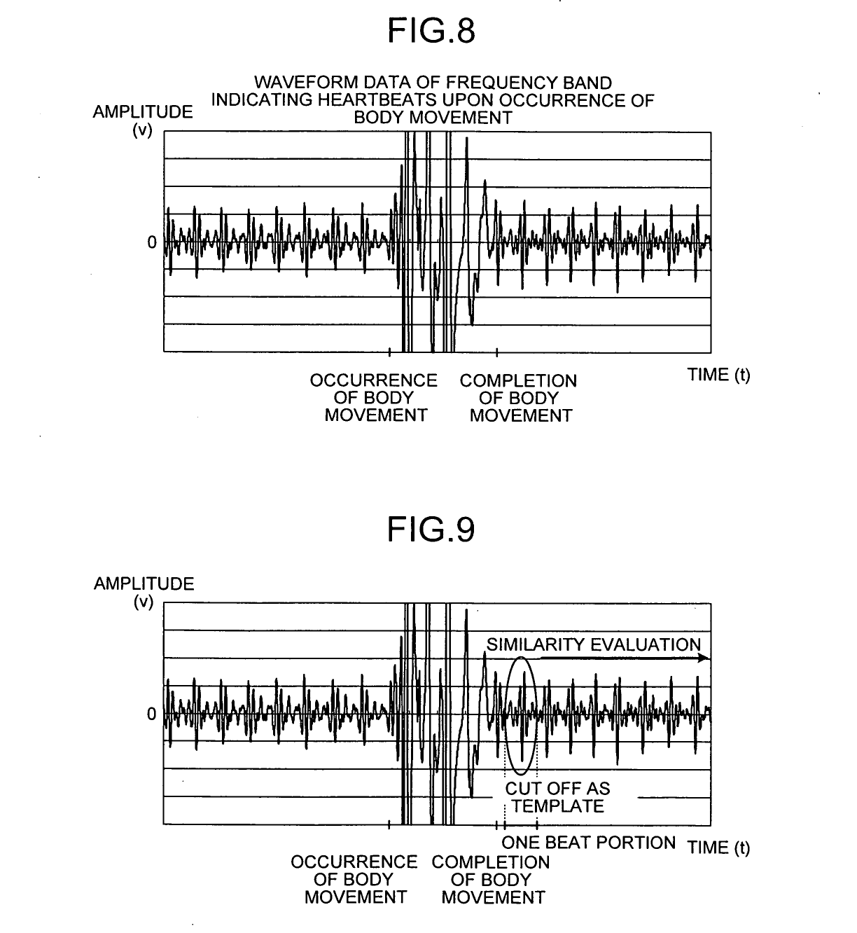

FIG. 8 shows one example of Waveform data of a frequency band indicating the heartbeat in accordance with biological information acquired by a biological information acquiring unit 101 upon occurrence of a body movement. As shown in this figure, it is confirmed that the waveform data of the heartbeat greatly fluctuate during a period from the occurrence of a body movement to the completion thereof. Therefore, upon detection of an amplitude greater than a predetermined amplitude, the body-movement detecting unit 102 regards this as the detection of a user's body movement. This predetermined amplitude is set based upon the amplitude of waveform data of heartbeats, which has been amplified by the biological amplifying unit 111, and from which excessive waveform data have been removed by the filter unit 112. For example, an amplitude that is 1.5 times the average amplitude of the heartbeat waveform is set as a predetermined amplitude, and when the body-movement detecting unit 102 detects an amplitude greater than this predetermined amplitude, it regards this amplitude as the detection of a body movement.

Moreover, after a lapse of a predetermined period of time since the amplitude exceeding the predetermined amplitude has not been detected from the waveform data of biological information, the body movement detecting unit 102 regards this condition as the completion of the body movement. This predetermined time is set as an appropriate period of time so as to determine that the body movement is completed based upon actual measurements, and, for example, is set to 5 seconds. In this manner, a fixed period of time is set in detail so as to detect a body movement; thus, the body movements can be detected with higher accuracy.

Since body movements can be detected through the above-mentioned processes by using the body-movement detecting unit 102, it becomes possible to detect a body movement without the necessity of installing a sensor for detecting body movements separately. Moreover, since both the heartbeat and body movements can be detected from waveform data of biological information, it becomes possible to reduce the number of processes in comparison with a system using separate sensor to detect body movements.

The function of the body-movement detecting unit 102 is not limited to detection of body movements from the amplitude of the heartbeat waveform, and the body-movement detecting unit 102 may be designed so that, when the waveform similarity, calculated in a waveform similarity calculating unit 104 that will be described later, drops drastically, this condition may be detected as the occurrence of a body movement, and so that, when the drastic drop of the waveform similarity has been stopped for a predetermined period of time or more, this condition may be detected as the completion of the body movement.

The template generating unit 103, which is provided with a heartbeat peak value detecting unit 113, is designed so that, upon start of measurements or upon completion of a body movement detected by the body movement detecting unit 102, based upon the time at which the peak of the heartbeat is detected by the heartbeat peak value detecting unit 113, the heartbeat waveform data with time intervals each corresponding one heartbeat is cut off to generate a template. Moreover, the template generating unit 103 is not necessarily required to always generate a template after each body movement, and when the previously generated template allows proper measurements of the heartbeat and the like with high accuracy, the previously generated template, as it is, may be used.

FIG. 9 is an explanatory drawing that shows an operation in which, by using, as a template, waveform data of heartbeats corresponding to one beat that have been cut off after the completion of the body movement in waveform data in the frequency band indicating heartbeats, the succeeding waveform data is evaluated on the similarity relationship. As shown in this figure, the template generating unit 103 cuts off waveform data indicating a heartbeat waveform corresponding to one beat from waveform data after the completion of a body movement so that, with respect to waveform data of heartbeats that is successively acquired by the biological information acquiring unit 101, the waveform similarity between the template and the waveform can be calculated, for example, as a correlation coefficient. A specific calculation method of the waveform similarity that is a numeric value indicating the similarity of waveforms will be explained later.

The heartbeat peak value detecting unit 113 calculates the peak value from waveform data of the heartbeat waveform upon start of measurements or upon completion of a body movement. Based upon the peak value of heartbeats detected by the heartbeat peak value detecting unit 113, the template generating unit 103 is allowed to cut off waveform data of heartbeats corresponding to one beat.

Moreover, the heartbeat peak value detecting unit 113 compares absolute values of a plurality of peak values generated as peak values in an upper direction and peak values in a downward direction of waveform data, and calculates a difference between the highest peak value and the second highest peak value in the same direction so that the direction having a greater difference is selected to detect the highest peak value in the selected direction. With this arrangement, the peak value to be compared is accurately determined so that it becomes possible to detect a peak value with higher accuracy. Here, the peak detection may be carried out, with the direction being fixed to either one of the upward and downward directions.

More specifically, the template generating unit 103 determines one peak among a plurality of heartbeat peaks detected by the heartbeat peak value detecting unit 113 as the center peak contained in the template, and cuts off an interval from the mid point between the peak contained in the template and the previous peak by one to the mid point between the peak contained in the template and the succeeding peak by one as one beat. Here, not limited to the interval from the mid point to the mid point, for example, an interval that is shorter than the interval from the mid point to the mid point may be cut off as the template as long as the peak is contained therein.

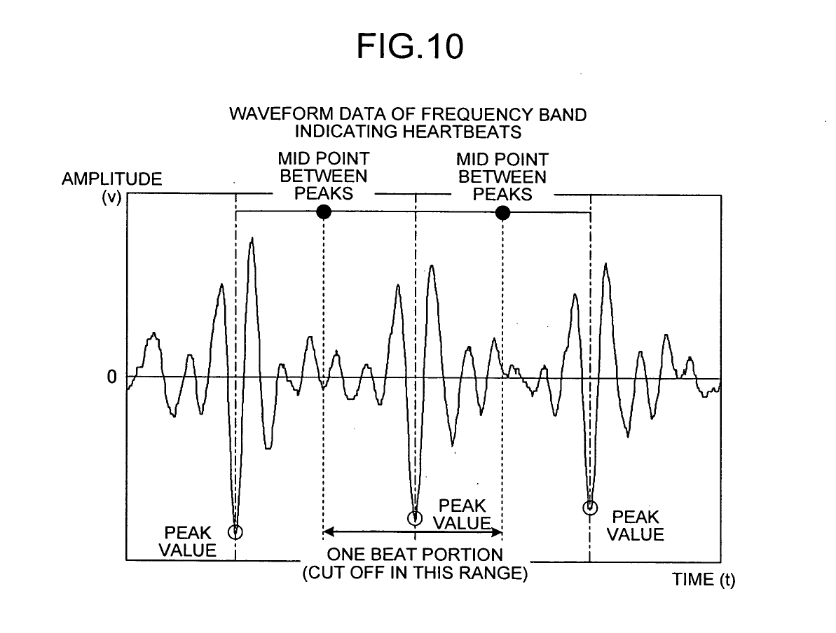

FIG. 10 is an explanatory drawing that shows an example in which based upon the peak value detected by the heartbeat peak value detecting unit 113, one beat portion is cut off from waveform data of a frequency area indicating heartbeats. By using the interval from the mid point to mid point between the peaks shown in this figure as one beat portion, the template generating unit 103 generates waveform data cut off from the range of this one beat portion as a template. When the template generating unit 103 generates the template in such a method, since the template is formed immediately after a body movement, it becomes possible to calculate heartbeats and also to analyze activities of the autonomic nerve system immediately after the body movement.

By using the template derived from waveform data generated by the template generating unit 103, the waveform similarity calculating unit 104 calculates the similarity relationship to the waveform data of biological information acquired by the biological information acquiring unit 101 as waveform similarity (for example, correlation coefficient). The waveform similarity thus calculated is used upon evaluating the template by using an evaluating unit 105, which will be described later, and is also used upon calculating the heart rate as well as analyzing the autonomic nerve system, after the template has been evaluated as an appropriate one by the evaluating unit 105.

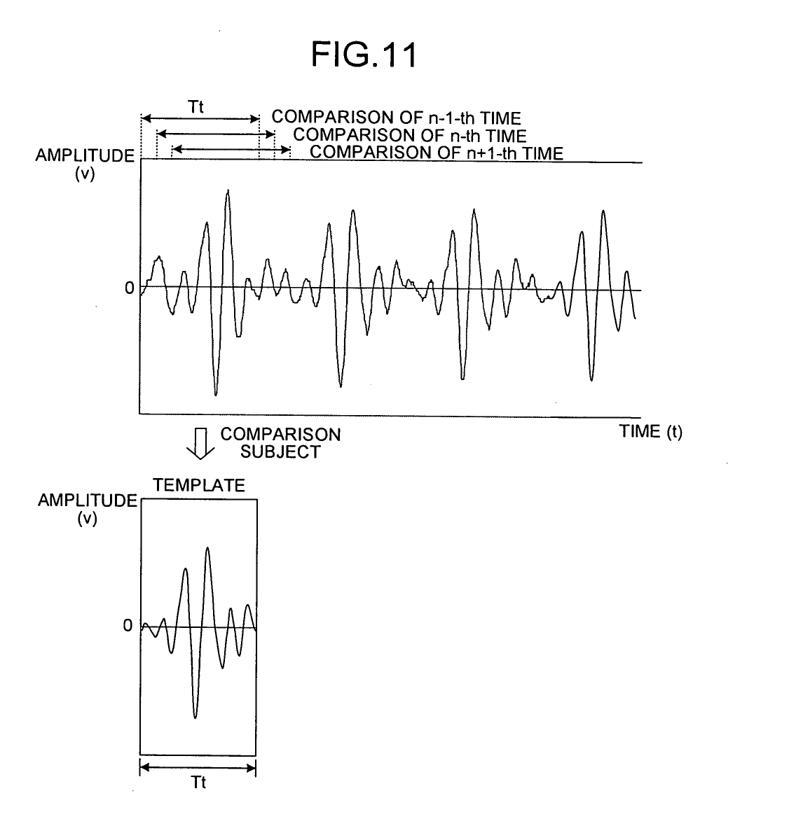

FIG. 11 is an explanatory chart that shows a sequence of processes in which the waveform similarity calculating unit 104 calculates the waveform similarity by using the template. The following description will discuss a case in which a correlation coefficient derived from self-correlation is used as the waveform similarity. As shown in this figure, supposing that the time interval of the template is Tt, the waveform data of the time interval Tt from a predetermined start time of waveform data of biological information acquired by the biological information acquiring unit 101 is compared with the template so that a correlation coefficient is calculated as the waveform similarity in which the similarity relationship of the waveform is indicated by a numeric value. After the correlation coefficient has been calculated, the waveform data in the time interval Tt from the time shifted from the predetermined start time by a predetermined sampling interval with respect to waveform data of biological information acquired by the biological information acquiring unit 101 is compared with the template so that a correlation coefficient is calculated. In the succeeding processes also, the same processes are carried out to calculate the correlation coefficient. Here, the correlation coefficient takes a value between −1 and 1, and is defined so that when the correlation coefficient takes a value of 1, the shape of the template is coincident with the shape of the waveform data of biological information, while when the correlation coefficient takes a value of −1, the shape of the template is completely opposite to the shape of the waveform data of biological information. Here, with respect to the method of calculating the waveform similarity, not limited to the above-mentioned method using the correlation coefficient, any method may be used as long as it allows the similarity relationship between the shape of the template and the shape of the waveform data of biological information to be calculated as a value.

After the template has been formed by the template generating unit 103, the evaluating unit 105 evaluates whether or not it is appropriate to carry out calculations on the heart rate and the like by using the generated template. In the present embodiment, the evaluating unit 105 evaluates whether or not the template is appropriate based upon the waveform similarity calculated by the waveform similarity calculating unit 104. Here, in the present embodiment, the evaluating process as to whether or not the template is appropriate is not intended to be limited to the determining process based upon the waveform similarity.

More specifically, when the number of waveforms in which the peak value per one beat of the correlation coefficient that is calculated as the waveform similarity by the waveform similarity calculating unit 104 within a predetermined evaluation determining time becomes lower than a predetermined evaluation determining value (defined as 0.7 in the present embodiment) is below a predetermined evaluation determining number (defined as 1 in the present embodiment), the evaluating unit 105 determines the corresponding template as an appropriate one, and the resulting template is used as a standard template. Here, the evaluation determining time is set as a period of time that is sufficient so as to evaluate the template, and in the present embodiment, a time interval corresponding to four heartbeats is set as the evaluation determining time.

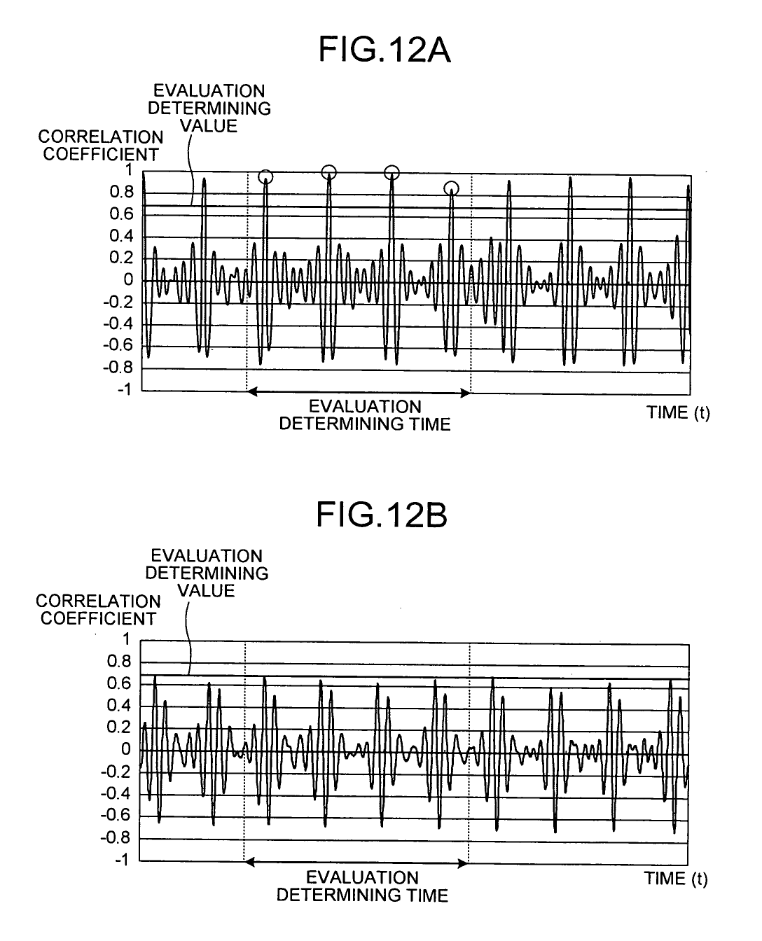

FIG. 12A shows a change in the correlation coefficient when the template is evaluated as an appropriate one by the evaluating unit 105. As shown in this figure, it is confirmed that during the evaluation determining time, all the peak values in the correlation coefficient exceed the evaluation determining value.

FIG. 12B shows a change in the correlation coefficient when the template is evaluated as an inappropriate one by the evaluating unit 105. As shown in this figure, since the number of times in which the peak value in the correlation coefficient becomes 0.7 or less in the evaluation determining value within the evaluation determining time is 1 or more in the number of evaluation determining times, the evaluating unit 105 evaluates the corresponding template as an inappropriate one.

When the template is evaluated as inappropriate by the evaluating unit 105, the template generating unit 103 newly generates a template again. Moreover, when the template is evaluated as appropriate by the evaluating unit 105, the succeeding processes are carried out by using the template that has been evaluated as an appropriate one based upon a system as described below. With these evaluation processes, the heartbeat measuring apparatus 100 of the present embodiment can carry out calculations on the heart rate and the like with improved accuracy.

After the template has been evaluated as appropriate by the evaluating unit 105, the peak-value detecting unit 106 detects a peak value and acquires a peak value time at which the peak value was obtained for each predetermined beat estimated time, that is, for each time period that is estimated as one beat portion, from the correlation coefficient calculated by the waveform similarity calculating unit 104 as the waveform similarity. With respect to the detection method used in the peak-value detecting unit 106, any detection method may be used, and, for example, a method in which a peak value of waveform data that has exceeded a threshold value is detected may be simply used. Here, the peak detected by the peak-value detecting unit 106 corresponds to a heartbeat of one time.

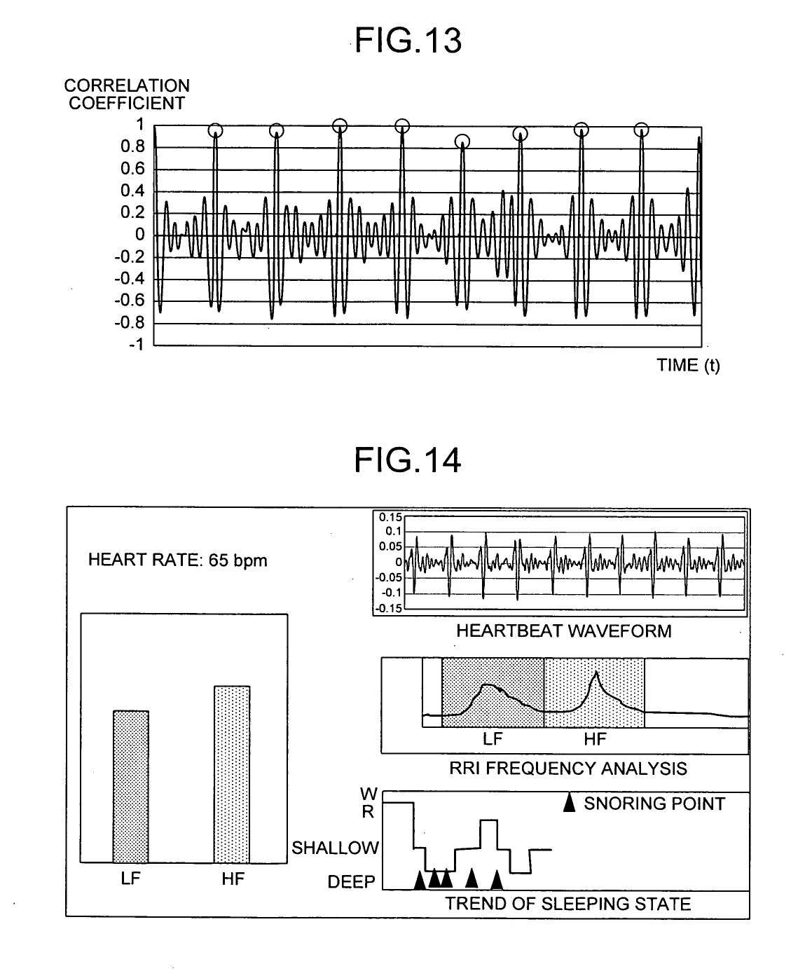

FIG. 13 shows one example of a correlation coefficient calculated by the waveform similarity calculating unit 104 as the waveform similarity, with a value forming a peak value detected by the peak value detecting unit 106 being enclosed by a circle. As shown in this figure, the peak-value detecting unit detects the peak value regularly.

Moreover, when the number of times at which the peak value, detected by the peak-value detecting unit 106, has exceeded a predetermined peak threshold value (for example, 0.7) within a predetermined body-movement detection time (for example, 10 seconds) becomes lower than a predetermined body-movement determination number of times (for example, 8 times), the body-movement detecting unit 102 to which this fact has been inputted from the peak-value detecting unit 106 determines that, although a big body movement is not occurring, the characteristics of waveform data of biological information detected by the sensor unit 10 have changed due to a change in the posture or the like. Therefore, upon determination that the characteristics of waveform data have changed, it is determined that there has been a body movement; thus, the same processes are again repeated from the generation of a template by the template generating unit 103.

A snore detecting unit 107 detects snoring from a frequency component that is presumably derived from snoring, which has been eliminated by the filter unit 112. With respect to a method of detecting snoring, any method may be used as long as it detects snoring from the high-frequency component that is presumably derived from snoring.

The heart rate calculating unit 108 calculates an instantaneous heart rate based upon the time interval of peaks obtained from the peak-value time acquired by the peak-value detecting unit 106. In other words, by dividing one minute by the time interval of the peaks, it becomes possible to calculate the instantaneous heart rate per minute. Moreover, the heart rate calculated by the heart rate calculating unit 108, as it is, may be outputted to a display processing unit 110, which will be described later; however, in order to stabilize a presented heart rate to be displayed on the display processing unit 110, another process, such as a moving average process or a process in which values that are greatly deviated from the previous instantaneous heart rate by a predetermined numeric value or more are ignored, may be carried out.

The autonomic nerve analyzing unit 109 frequency-analyzes the time interval of peaks, obtained from the peak-value time acquired by the peak-value detecting unit 106, and analyzes activities of the autonomic nerve system based upon peaks (LF) appearing in a range of 0.03 Hz to 0.1 Hz and peaks (HF) appearing in a range of 0.1 Hz to 0.5 Hz.

Here, HF represents a value that reflects the active state of the parasympathetic nerve of the autonomic nerve system, and LF represents a value that mainly reflects the active state of the sympathetic nerve of the autonomic nerve system, although it is modified by the parasympathetic nerve. Moreover, from the activity by the autonomic nerve system, it is possible to estimate a sleeping state to a certain degree. For example, depending on whether or not HF is greater than a first predetermined value, whether a sleeping state is an NREM sleeping state or a REM sleeping state is specified, and when it is specified as the NREM sleeping state, it is proposed to specify whether the sleeping state is a deep sleeping state or a shallow sleeping state, by further making a comparison as to whether or not it is greater than a second predetermined value. Here, the first predetermined value and the second predetermined value are set based upon actual measurements, because these values are different depending on the users.

The display processing unit 110 carries out displaying processes of the heart rate calculated by the heart rate calculating unit 108 and the results of analyses by the autonomic nerve analyzing unit 109 in appropriate modes. With respect to the display end of the display processing unit 110, any device may be used, and, although not shown in FIG. 1, for example, a liquid crystal panel attached to a mat-type device or a pillow-type device, a wrist watch-type display unit equipped with an accelerometer and the like, or a monitor, are proposed. Moreover, the display processing unit 110 displays information of snoring detected by the snore detecting unit 107 together with information of a sleeping state. With this arrangement, the user is allowed to confirm the state of snoring during the sleeping state in an objective manner.

FIG. 14 shows an example of a screen displayed on a monitor 40, which includes the results of processes carried out by a display processing unit 110. As shown in this figure, the following data are displayed on the monitor 40: the heart rate calculated by the heart rate calculating unit 108, the waveform data that indicates heartbeats acquired by the biological information acquiring unit 101, the results obtained by frequency-analyzing the intervals between peaks of the correlation coefficient analyzed by the autonomic nerve analyzing unit 109, HF and LF obtained as the results of the frequency analysis, and the sleeping state and the like obtained by HF and LF. Moreover, the snoring information detected by the snore detecting unit 107 is displayed on the sleeping state. With this arrangement, the sleeping state can be diagnosed in more detail.

In the present embodiment, both the heart rate calculating unit 108 and the autonomic nerve analyzing unit 109 are prepared; however, both of these are not necessarily required, and only one of these may be installed depending on the purpose.

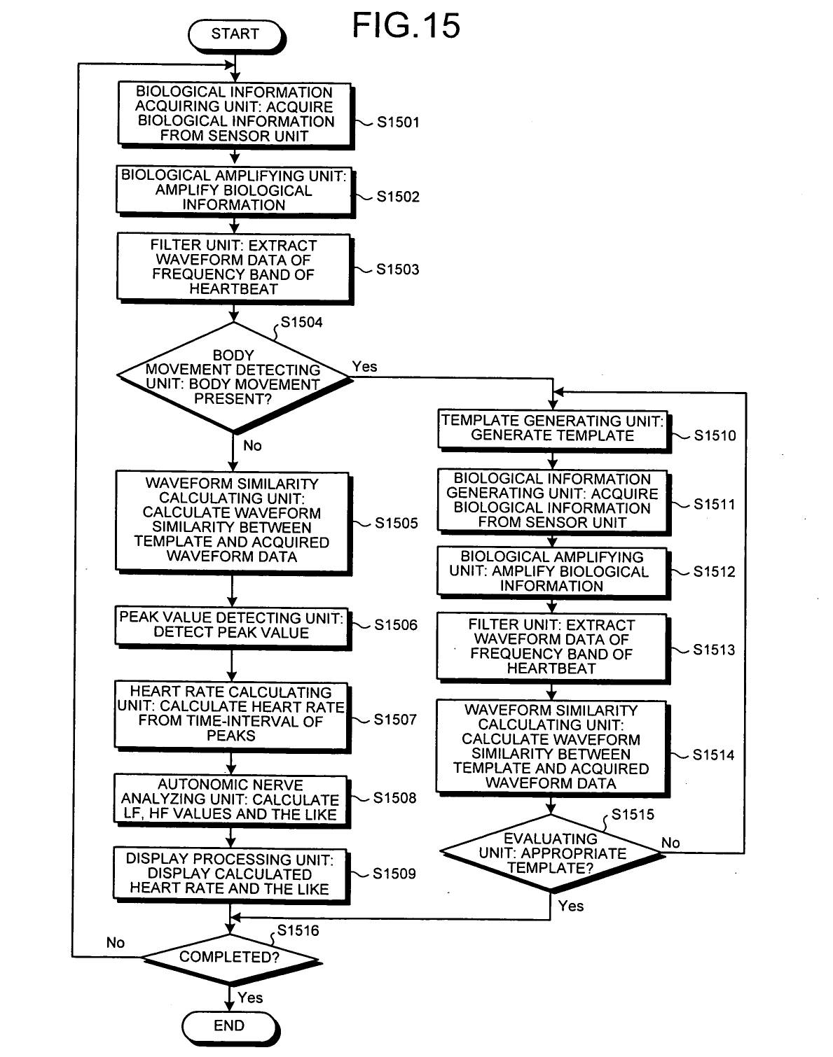

Next, the following description will discuss processes in which, in the heartbeat measuring apparatus 100 in accordance with the present embodiment constructed as described above, based upon biological information detected by the sensor unit 10, the heart rate is calculated and LF, HF values and the like are calculated so that the results thereof are displayed on the monitor 40. FIG. 15 is a flow chart that shows a sequence of the above-mentioned processes carried out by the heartbeat measuring apparatus 100 in accordance with the present embodiment.

First, the biological information acquiring unit 101 acquires a signal inputted from the sensor unit 10 as biological information (step S1501). Next, the biological amplifying unit 111 amplifies the acquired biological information (step S1502). Moreover, the filter unit 112 extracts waveform data of a frequency band indicating heartbeats from the amplified biological information (step S1503).

Based upon the waveform data of heartbeats thus extracted, the body-movement detecting unit 102 detects whether or not any body movement is occurring (step S1504). Here, the body-movement detecting unit 102 detects any body movement based upon whether or not the amplitude of heartbeats is greater than a predetermined amplitude.

When the body-movement detecting unit 102 has detected a body movement (Yes in step S105), after determining that the body movement has been completed, the template generating unit 103 generates a template (step S1510). Here, the template generating unit 103 cuts off waveform data in a time interval corresponding to one heartbeat based upon the time at which the peak of the heartbeats was detected by the heartbeat peak value detecting unit 113, and forms the template. In the present embodiment, a template is generated at the start of measurements of heartbeats, even when no body movement has been detected.

After the template has been generated, the biological information acquiring unit 101 acquires a signal inputted from the sensor unit 10 as biological information (step S1511). Next, in the same manner as the sequence of processes of steps S1502 and S1503, the biological information acquired by the biological amplifying unit 111 is amplified so that the filter unit 112 extracts waveform data of a frequency band indicating heartbeats from the amplified biological information (steps S1512 and S1513).

Next, the waveform similarity calculating unit 104 calculates the waveform similarity (for example, correlation coefficient) that indicates a similarity relationship between the template generated by the template generating unit 103 at step S1510 and the waveform data of a frequency band indicating heartbeats extracted at step S1513 (step S1514).

Moreover, based upon the correlation coefficient calculated as the waveform similarity, the evaluating unit 105 evaluates whether or not the generated template is appropriately used for measuring heartbeats and the like (step S1515). In the present embodiment, when the number of waveforms in which the peak value for each beat in the correlation coefficient calculated by the waveform similarity calculating unit 104 becomes 0.7 or less in the evaluation determining value within a predetermined evaluation determining time is 1 or less in the number of evaluation determining times, the evaluating unit 105 evaluates the corresponding template as an appropriate one.

When a template has been evaluated by the evaluating unit 105 as an inappropriate one (No in step S1515), the template generating unit 103 again generates a template based upon waveform data of a frequency band indicating heartbeats acquired through steps S1511 to S1513 (step S1510), and the processes up to evaluation as to whether or not the template is appropriate are then carried out (steps S1511 to S1515).

When a template has been evaluated by the evaluating unit 105 as an appropriate one (Yes in step S1515), the processes are not completed unless the user leaves the sensor 10 (No in step S1516), the processes are restarted from the acquiring process of biological information by the biological information acquiring unit 101 (step S1501). Here, with respect to the case in which the user leaves the sensor unit 10, the description thereof will be given later.

When the body-movement detecting unit 102 has not detected any body movement (No in step S1504), the waveform similarity calculating unit 104 calculates the waveform similarity (for example, correlation coefficient) that indicates a similarity relationship between the template generated at step S1510 and the waveform data extracted at step S1503 (step S1505).

Moreover, the peak value detecting unit 106 detects a peak value from the correlation coefficient calculated by the waveform similarity calculating unit 104, and acquires peak value time at which the peak value appeared (step S1506).

The heart rate calculating unit 108 calculates an instantaneous heart rate based upon a time interval between peaks obtained from the peak value time acquired by the peak value detecting unit 106 (step S1507). Next, the autonomic nerve analyzing unit 109 frequency-analyzes the time interval between peaks obtained from the peak value time acquired by the peak value detecting unit 106 in the same manner so that the autonomic nerve is analyzed; thus, more specifically, LF and HF values are calculated and the sleeping state is estimated (step S1508).

The display processing unit 110 carries out processes so as to display data, such as the calculated heart rate, calculated LF and HF values and estimated sleeping state, on the monitor 40 (step S1509).

When the user has risen and left the sensor unit 10, the heartbeat measuring apparatus 100 determines that measurements are no longer available, thereby completing the processes (Yes in step S1516). In contrast, when the user does not leave the sensor unit 10, the heartbeat measuring apparatus 100 determines that the measuring processes are continuously carried out (No in step S1516), and again starts the processes from step S1501.

Through the above-mentioned sequence of processes, the template is formed from the acquired biological information, and it becomes possible to analyze the heart rate and the autonomic nerve by using the template thus formed. Here, the above-mentioned sequence of processes have exemplified a sequence of processes in the present embodiment from the acquisition of biological information to the display of the calculated heart rate and the like, and the present invention is not intended to be limited by this sequence of processes.

Moreover, in the above-mentioned sequence of processes, the heartbeat measuring apparatus 100 carries out both the calculations of the heart rate and LF, HF values and the like from the detected biological information; however, both of these are not necessarily required to be calculated, and only one of these may be calculated. Furthermore, although the description thereof is omitted in the above-mentioned flow chart, the heartbeat measuring apparatus 100 of the present invention is designed so that the snore detecting unit 107 detects snoring based upon a high-frequency component removed by the filter unit 112 at step S1503.

In the above-mentioned embodiment, the heart rate calculation and the autonomic nerve analysis are carried out based upon heartbeats; however, not limited to the heartbeats, the above-mentioned processes may be carried out based upon measured pulses so as to conduct the heart rate calculation and the autonomic nerve analysis.

Moreover, in the above-mentioned embodiment, the correlation coefficient is set to a value from −1 to 1; however, the value to which the correlation coefficient is set is not intended to be limited to a value in this range, and any value is used as long as the similarity relationship between the waveform data of acquired biological information and the waveform data of the template can be recognized.

In the present embodiment, the evaluation determining time is set to a time interval corresponding to four heartbeats or more and the evaluation determining value is obtained at 0.7 in the correlation value with the evaluation determining number of times being set to 1; however, the present invention is not intended to be limited by these values, and any appropriate values can be set so as to determine whether or not the template is appropriate.

In the heartbeat measuring apparatus of the present embodiment, a template suitable for the state of the user after a body movement is formed, and heart rate calculations and autonomic nerve analyses are carried out based upon an interval between peaks of the correlation coefficient calculated by using the template thus formed; therefore, even when a change in the waveform occurs after a body movement, it becomes possible to carry out heart rate calculations and autonomic nerve analyses with high accuracy. Moreover, since heart rate calculations and autonomic nerve analyses are carried out by using a template that has been evaluated by the evaluating unit 105 as an appropriate one, it becomes possible to carry out heart rate calculations and autonomic nerve analyses with high accuracy.

In the first embodiment, the body-movement detecting unit 102 detects a body movement based upon the amplitude of a frequency band indicating heartbeats; however, the present invention is not intended to be limited by the body-movement detection means of this type. Therefore, the second embodiment will discuss a system in which the body movement is detected by using a sensor different from the sensor unit 10 that acquires a living body signal.

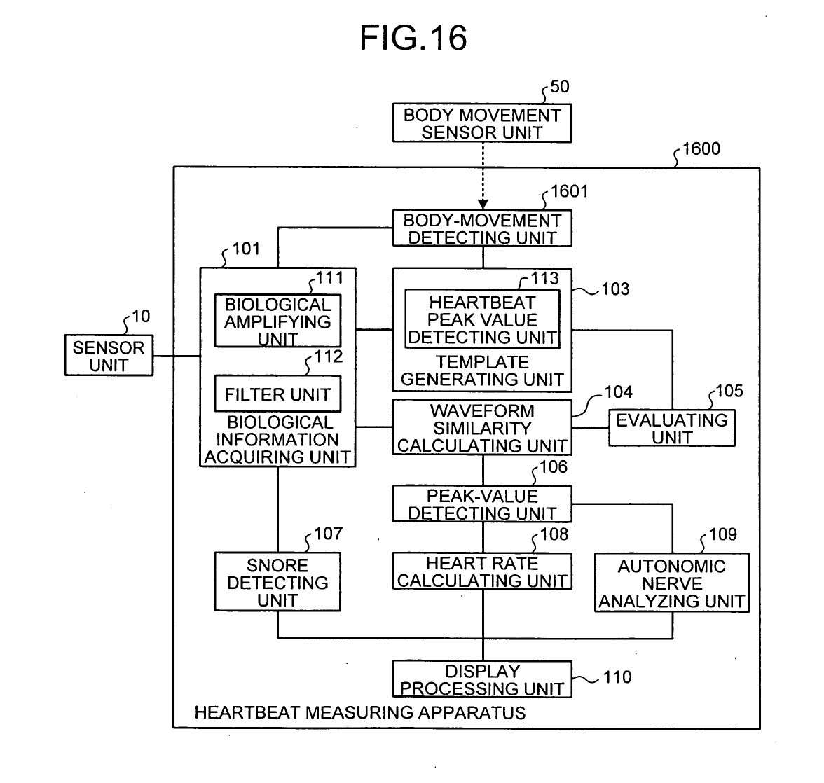

FIG. 16 is a block diagram that shows a structure of a heartbeat measuring apparatus 1600 in accordance with the second embodiment. The system of the second embodiment is different from the heartbeat measuring apparatus 100 of the above-mentioned first embodiment in that the body-movement detecting unit 102 is changed to a body-movement detecting unit 1601 that carries out different processes. In the following description, the same constituent parts as those of the above-mentioned first embodiment are indicated by the same reference numerals, and the description thereof is omitted.

Based upon a signal inputted from a body-movement sensor unit 50 attached to the user, the body-movement detecting unit 1601 detects whether or not any body movement is occurring. In the present embodiment, the body-movement sensor unit 50 is prepared as an accelerometer, which is attached to the user's body so as to directly detect body movements. With respect to the attaching portion, although not particularly limited, the sensor unit is attached to the arm in the present embodiment, and based upon a signal inputted to the heartbeat measuring apparatus 1600 from the attached body-movement sensor unit 50 through a radio communication unit or the like, the body movement detecting unit 1601 detects a body movement. Additionally, the communication unit is omitted from FIG. 16.

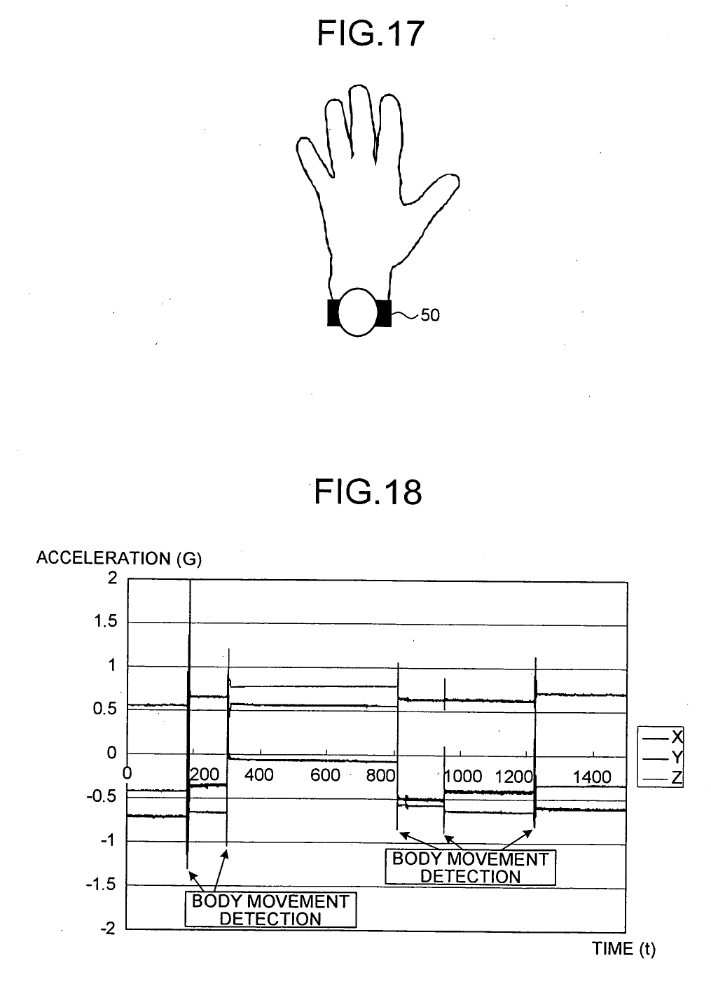

FIG. 17 shows one example of the body-movement sensor unit 50 attached to the user. The body-movement sensor 50 is assembled into a main body of a wrist watch type, shown in this figure, so that the body movements can be detected. Here, when the output level of a signal inputted from the body-movement sensor unit 50 is high, it is neither necessary to amplify the level by using an amplifier, nor necessary to remove a predetermined frequency band by using a filter.

FIG. 18 shows one example of a signal that is outputted when a 3-axis accelerometer is used as the body-movement sensor unit 50. In the present figure, the axis of abscissa indicates second, and the axis or ordinates indicates gravitational acceleration (G). When the gravitational accelerations of all the axes greatly fluctuate instantaneously, the body-movement detecting unit 1601 detects these as a body movement. For example, when the fluctuation of acceleration of each of the axes exceeds a predetermined value (for example, ±0.2 G) within a predetermined period of time (for example, 1 second), the body-movement detecting unit 1601 determines this state as a detection of a body movement.

Moreover, an attaching-type pulse wave sensor to measure heartbeats or pulses, which is different from the present invention, may be used in place of the mat-type sensor unit 10, and the body-movement sensor unit may be incorporated into this attaching-type pulse wave sensor. For example, the biological information acquiring unit 101 acquires a signal derived from pulse waves on the wrist portion or palm portion measured by the main body of the wrist watch type as shown in FIG. 17 as biological information, and the same processes as those of the first embodiment are carried out so that heart rate calculations and autonomic nerve analyses can be carried out. Furthermore, by incorporating the body-movement sensor unit into the attaching-type pulse wave sensor, it becomes possible to detect body movements. Thus, since the pulse wave sensor and the body-movement sensor unit are allowed to acquire signals from the same portion of the same user, it is possible to improve the accuracy.

Here, the sequence of processes to be carried out by the heartbeat measuring apparatus 1600 in accordance with the present embodiment arranged as described above are the same as those of the first embodiment except that the processes of the body-movement detecting unit 1601 for detecting body movements are different from those of the body-movement detecting unit 102; therefore, the description thereof is omitted.

As indicated by the heartbeat measuring apparatus 1600 of the present embodiment, even in the system using a plurality of sensors, heart rate calculations and autonomic nerve analyses can be carried out. Moreover, since the body-movement detecting unit 102 is designed to detect a body movement based upon a signal inputted from the body-movement sensor unit 50, the body-movement sensor unit 50 is attached to a portion suitable for detecting body movements so that it becomes possible to detect body movements with high accuracy.

Moreover, the present invention is not intended to be limited by the above-mentioned embodiments, and various modifications, for example, as shown below, may be made.

In the above-mentioned embodiment, in order to generate a template, the template generating unit 103 specifies a mid point between peaks so as to cut off waveform data corresponding to one beat portion from heartbeat data, and cuts off waveform data from the mid point to the succeeding mid point by one as one beat portion. However, in the above-mentioned embodiment, the process is not intended to be limited by this cutting-off process of the waveform data. Therefore, in a template generating unit of a first modification, for example, a time interval R of two peaks is measured, and based upon the heartbeat peak detected by the heartbeat peak value detecting unit, a range of ±R/2 or less is cut off as one beat portion.

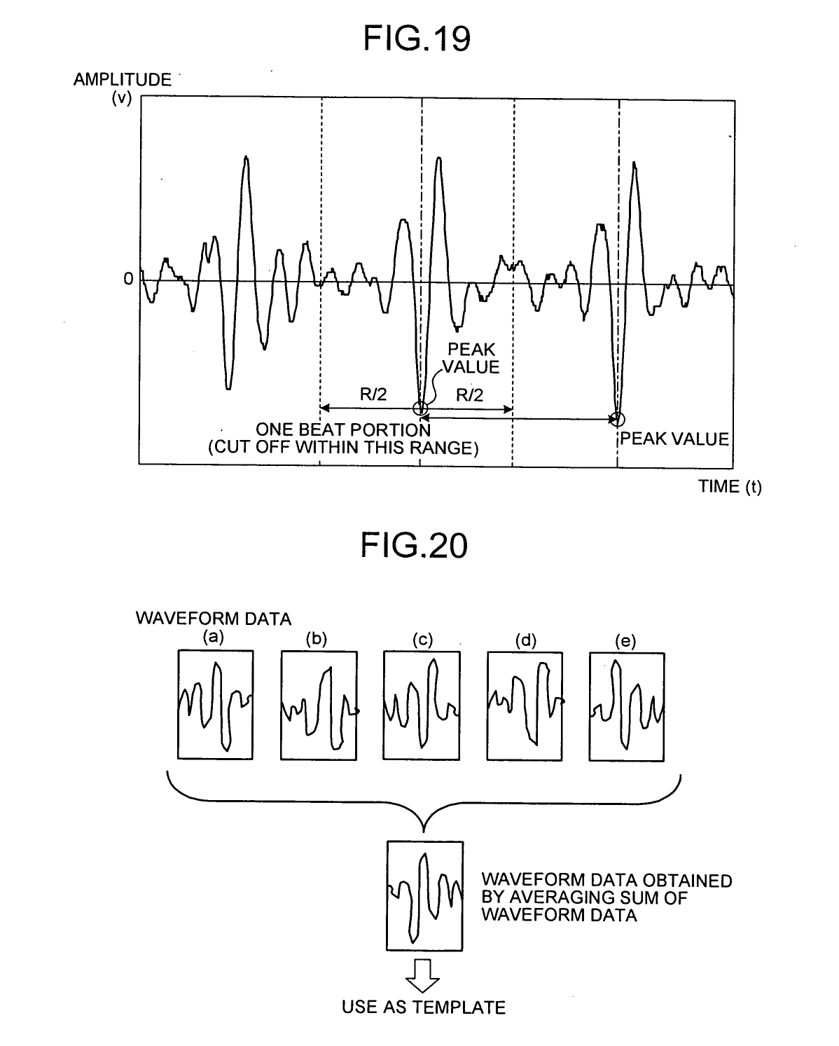

FIG. 19 is an explanatory drawing that shows an example in which, in a modification, based upon a peak value detected by a heartbeat peak value detecting unit 113, one beat portion is cut off from waveform data of a frequency area indicating the heartbeat. As shown in this figure, after specifying an interval R between two peaks, the template generating unit of the present modification cuts off a range of ±R/2 as waveform data corresponding to one beat portion based upon one peak detected by the heart-beat peak value detecting unit so that a template is formed.

The use of the cutting-off process of one beat portion shown in the present modification allows to specify a time interval corresponding to one beat portion simply by detecting two peaks so that it becomes possible to cut off waveform data corresponding one beat portion quickly and also to reduce the processes required for the cutting-off process. Additionally, a plurality of intervals each of which corresponds to an interval from the preceding peak by one or an interval to the succeeding peak by one may be obtained, and the average of these may be defined as R.

In the above-mentioned embodiments, with respect to the method of generating the template, waveform data corresponding to one beat portion, first cut off after the completion of a body movement, is generated as a template. However, the above-mentioned embodiments are not intended to be limited by the above-mentioned template generation method, and any method may be used as long as a template of waveform data of heartbeats can be formed after the detection of a body movement. Therefore, in the second modification, after the template generating unit has obtained a plurality of waveform data, each corresponding to one heartbeat portion, it calculates waveform data that forms an average value of these so that a template is generated.

FIG. 20 is an explanatory drawing that indicates a sequence of processes in which a template generating unit calculates waveform data that is used as a template from a plurality of acquired waveform data corresponding to one beat portion of the heartbeat. As shown in this figure, the template generating unit cuts off a plurality of waveform data corresponding to one beat portion from the biological information acquired by the biological information acquired unit in the same sequence of processes as those of the above-mentioned embodiment. With respect to the plurality of waveform data, each corresponding to one beat portion of the heartbeat, the template generating unit adds these waveform data based upon the peak, and obtains the averaged waveform data as a template.

In comparison with the processes of the aforementioned embodiment, the processes shown in the present modification are designed to cut off a plurality of waveform data and carry out predetermined processes thereon so that a template is generated; therefore, although more time is required in comparison with the processes for generating the template of the aforementioned embodiment, the validity of the template can be improved because the template is generated based upon more waveform data.

Here, any method may be used as long as it generates a template of waveform data for heartbeats after the detection of a body movement as described above. Therefore, in a third modification, after acquiring a plurality of waveform data, each corresponding to one beat portion of the heartbeats, upon forming a template, the template generating unit selects one waveform datum as a subject for use in comparison, and calculates the waveform similarity between this selected waveform datum and another waveform data; and, after removing waveform data having not more than a predetermined waveform similarity (for example, 0.7) from this, the template generating unit further calculates waveform data that forms an average value of a plurality of waveform data so that a template is formed. Here, with respect to the method of calculating the similarity, any method may be used.

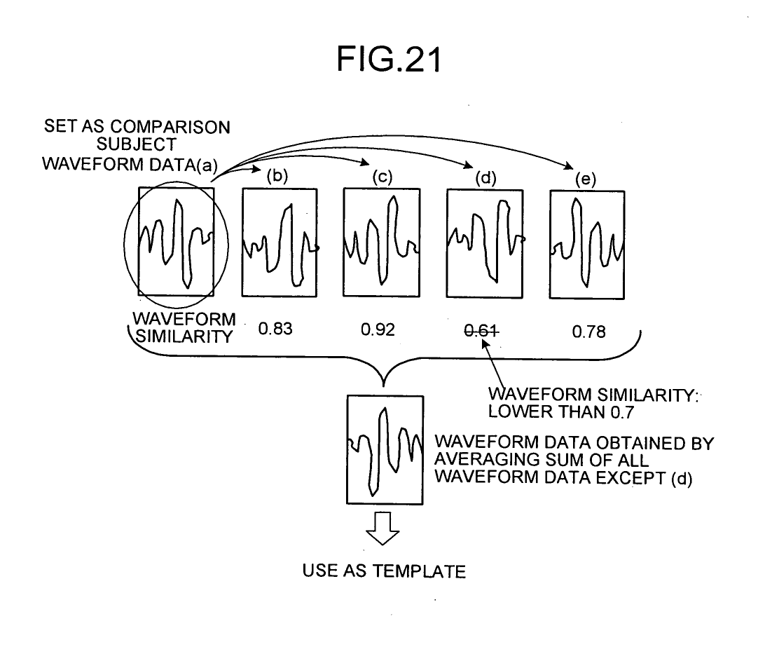

FIG. 21 is an explanatory drawing that indicates a sequence of processes in which a template generating unit acquires waveform data that is used as a template from a plurality of acquired waveform data corresponding to one beat portion of the heartbeat. As shown in this figure, the template generating unit cuts off a plurality of waveform data, each corresponding to one beat portion of the heartbeats, from biological information acquired by the biological information acquiring unit, in the same sequence of processes as those of the above-mentioned embodiment. Moreover, the template generating unit selects the first waveform datum (a) of the plurality of waveform data, each corresponding to one beat portion of the heartbeats, thus cut off, as the subject for use in comparison, and calculates the waveform similarity to each of the other waveform data (b) to (e). Then, the waveform data (d) having a value of 0.7 or less in the waveform similarity thus calculated is removed, and the waveform data (a), (b), (c) and (e) are added based upon the peaks of these waveform data so that the averaged waveform data is used as a template.

The waveform data, formed in the template generating unit of the present modification, is obtained by calculating the averaged waveform data after having removed waveform data that is low in the waveform similarity; therefore, the validity of the template is further improved in comparison with the template formed in the second modification. Here, the waveform data for use in comparison is not intended to be limited by the first waveform data, and any waveform data may be used as long as it corresponds to cut off waveform data corresponding to one beat portion of the heartbeat.

Here, any method may be used as long as it is used for generating a template of waveform data of heartbeats after the detection of a body movement, as described above. Therefore, in a fourth modification, after acquiring a plurality of waveform data, each corresponding to one beat portion of the heartbeats, upon forming a template, the template generating unit finds the sum of calculated waveform similarities of all the other waveform data with respect to the respective waveform data thus acquired, and selects the waveform data having the highest sum of the waveform similarities so that a template is formed.

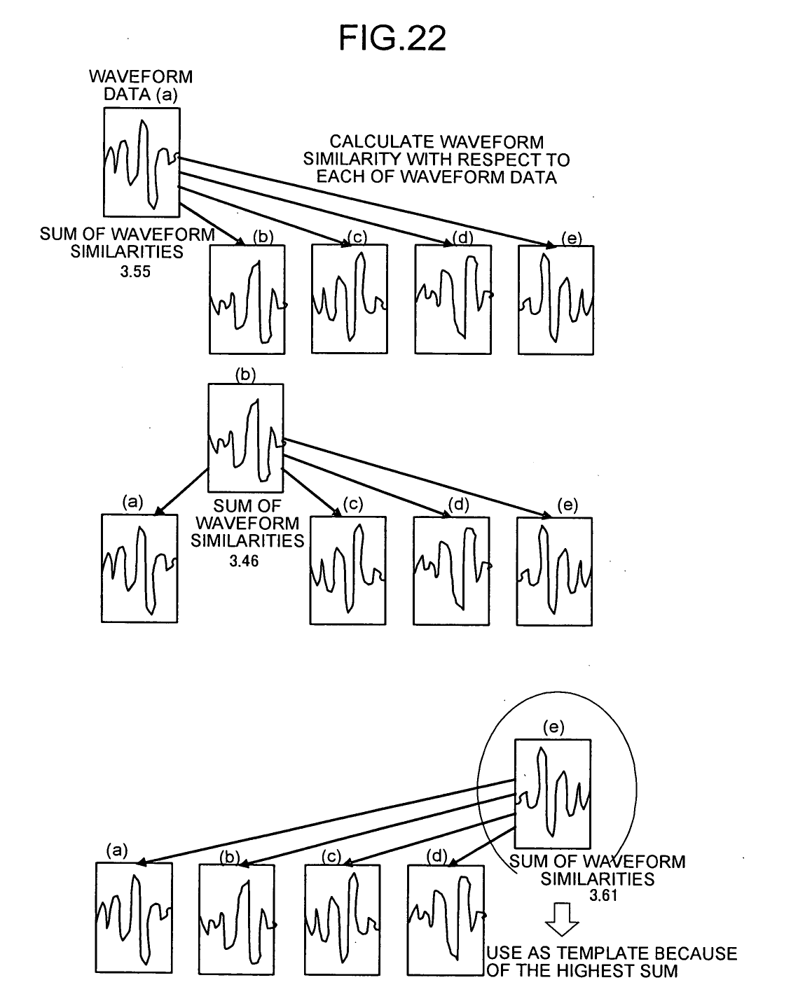

FIG. 22 is an explanatory drawing that indicates a sequence of processes in which the template generating unit selects waveform data that is used as a template from a plurality of acquired waveform data, each corresponding to one beat portion of the heartbeat. As shown in this figure, the template generating unit cuts off a plurality of waveform data, each corresponding to one beat portion, from biological information acquired by the biological information acquiring unit, in the same sequence of processes as those of the above-mentioned embodiment. Moreover, the template generating unit compares each of the waveform data thus cut off with all the other waveform data to calculate waveform similarities. For example, in the case of the first waveform data (a), the waveform data (a) is compared with all the other waveform data (b) to (e) to calculate the respective waveform similarities. Then, the sum of these waveform similarities is found. In the example shown in this figure, the sum of the waveform similarities of the waveform data (a) becomes 3.55. With respect to each of the waveform data (b) to (e), the sum of the waveform similarities is found in the same sequence of processes. Thus, the waveform data having the highest sum of the waveform similarities is selected as a template. In the example shown in this figure, since the sum of the waveform similarities of the waveform data (e) is highest, the template generating unit selects the waveform data (e) as a template.

In the present modification, in the same manner as the second and the third modifications, although it takes long time to generate the template, since the best suited waveform data is selected from the candidate waveform data, it becomes possible to improve the validity of the template generated by the template generating unit in the present modification.

Moreover, the process for evaluating whether or not the generated template is appropriate is not intended to be limited to the process for evaluation carried out by the evaluating unit 105 in the above-mentioned embodiment. Therefore, in a fifth modification, upon evaluating whether or not the template is appropriate, the evaluating unit carries out calculations in such a manner that, with respect to correlation coefficients calculated as the waveform similarities in the waveform similarity calculating unit within a time interval corresponding to one beat portion of the heartbeats, a plurality of magnifications of the highest peak value to the next highest peak value are calculated so that evaluation as to whether or not a template is appropriate is made based upon whether or not the average magnification of these is a predetermined multiple or more (for example, two times). In other words, when the average magnification thus calculated is 2 times or more, the evaluating unit determines that the corresponding template is appropriate.

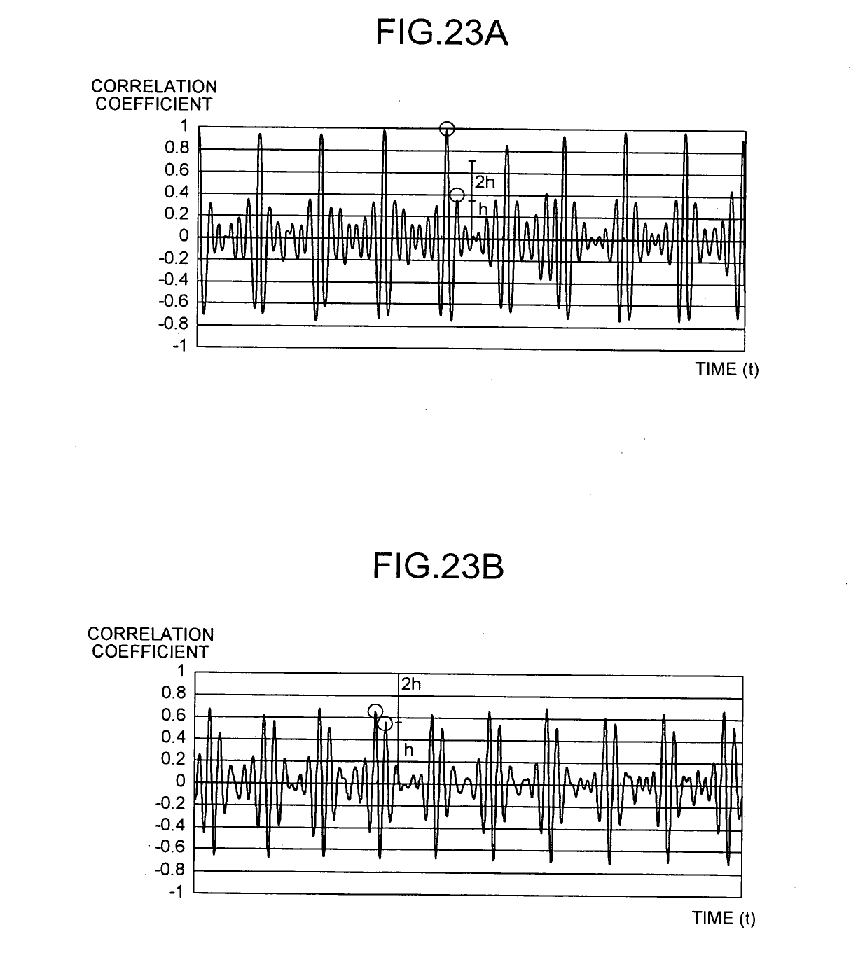

FIG. 23A shows a change in the correlation coefficient when the template is evaluated as an appropriate one by the evaluating unit of the present modification. As shown in this figure, supposing that the second highest peak value is “h” within a time interval corresponding to one beat portion, the highest peak value becomes “2h” or more; therefore, the corresponding template is evaluated as an appropriate one.

FIG. 23B shows a change in the correlation coefficient when the template is evaluated as an inappropriate one by the evaluating unit of the present modification. As shown in this figure, supposing that the second highest peak value is “h” within a time interval corresponding to one beat portion, the highest peak value becomes “2h” or less; therefore, the corresponding template is evaluated as an inappropriate one.

In the evaluating unit of the present modification, the highest peak value is set to 2 times or more of the second highest peak value; therefore, it becomes possible to prevent a plurality of peaks within a time interval for one beat portion from being detected as a plurality of heartbeats, and consequently to improve the reliability of the apparatus. Here, with respect to the process for evaluating the template, not limited to the present modification, any method may be used.

As described above, the heartbeat measuring device and the heartbeat measuring method in accordance with the present invention are effectively used for measuring the heart rate of the user, and in particular, appropriately applied as a technique for accurately measuring the heart rate even after a body movement of the user.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A heartbeat measuring apparatus comprising:
an biological information acquiring unit that acquires biological information derived from heartbeats, pulses or the like of a user;
a body-movement detecting unit that detects a body movement of the user;
a reference information generating unit that generates reference information, which serves as a reference indicating the heartbeats or pulses for comparison, based upon the biological information acquired by the biological information acquiring unit after completion of a detection of the body movement;
a waveform similarity calculating unit that calculates a waveform similarity between the reference information and biological information acquired after the reference information is generated;
an extreme value acquiring unit that specifies an extreme value of the heartbeats or pulses from a plurality of waveform similarities, and acquires extreme value time at which the extreme value is obtained; and
an biological information analyzing unit that analyzes biological information of the user based upon a time interval between the extreme value times.

2. The heartbeat measuring apparatus according to claim 1, wherein the biological information analyzing unit includes a heart rate calculating unit which calculates heart rate based upon the time interval between the extreme value times.

3. The heartbeat measuring apparatus according to claim 1, wherein the biological information analyzing unit includes an autonomic nerve analyzing unit that analyzes activities of autonomic nerve based upon the time interval between the extreme value times.

4. The heartbeat measuring apparatus according to claim 1, wherein, the body-movement detecting unit determines that there is a body movement of the user when waveform information of the biological information has a predetermined amplitude or more.

5. The heartbeat measuring apparatus according to claim 1, wherein the body-movement detecting unit determines that there is a body movement of the user when the extreme value does not exceed a predetermined body-movement determining value during a predetermined body-movement detection time equal to or more times than the number of predetermined body-movement determination operations.

6. The heartbeat measuring apparatus according to claim 1, wherein the body-movement detecting unit detects a body movement based upon an acceleration signal supplied from an acceleration detecting unit which is attached to a living body of the user.

7. The heartbeat measuring apparatus according to claim 1, further comprising:
an evaluating unit that evaluates whether or not the reference waveform information is appropriate based upon the calculated waveform similarity,
wherein the extreme value acquiring unit acquires the extreme value time, at which the extreme value is obtained, by specifying the extreme value of the heartbeats or pulses based on the plural waveform similarities, when the reference information is evaluated as being appropriate.

8. The heartbeat measuring apparatus according to claim 7, wherein the reference information generating unit generates another piece of the reference information which serves as a reference indicating the heartbeats or pulses for comparison based upon the biological information acquired by the biological information acquiring unit after acquiring the biological information employed for the generation of the reference information, when the reference information is evaluated as being inappropriate.

9. The heartbeat measuring apparatus according to claim 7, wherein the evaluating unit evaluates whether or not the reference information is appropriate based upon whether or not an extreme value, at which the waveform similarity calculated by the waveform similarity calculating unit is the highest during a time interval corresponding to one heartbeat, is equal to or lower than a predetermined evaluation determining value within a predetermined evaluation determining time equal or more times than the number of a predetermined evaluation determination operations.

10. The heartbeat measuring apparatus according to claim 7, wherein the evaluating unit evaluates whether or not the reference information is appropriate based upon whether or not an average value of the extreme values at which the calculated waveform similarity is the highest during a time interval corresponding to one heartbeat is equal to or higher than a predetermined evaluation determining multiple determined with respect to an average value of extreme values at which the waveform similarity is second highest.

11. The heatbeat measuring apparatus according to claim 7, wherein the evaluating unit evaluates whether or not the reference information is appropriate based upon whether or not an extreme value at which the waveform similarity calculated by the waveform similarity calculating unit is the highest during a time interval corresponding to one heartbeat is equal to or lower than a predetermined evaluation determining value within a predetermined evaluation determining time equal or more times than the number of a predetermined evaluation determination operations, even when the reference information is evaluated as being appropriate based upon the waveform similarity calculated by the waveform similarity calculating unit and employed in processing.

12. The heartbeat measuring apparatus according to claim 1, wherein the biological information acquiring unit includes a removing unit that removes a portion of the biological information from the biological information, which is acquired from a living body of the user by the sensor unit, the portion having a band other than a predetermined band which indicates heartbeats or pulses, to acquire the biological information based upon the predetermined band which indicates heartbeats or pulses.

13. The heartbeat measuring apparatus according to claim 1, wherein the reference information generating unit cuts out a time interval corresponding to one heartbeat or a pulse based upon the acquired biological information, and generates the reference information.

14. The heartbeat measuring apparatus according to claim 13,
wherein the reference information generating unit includes a heartbeat extreme value detecting unit that detects an extreme value of an amplitude of the waveform information of heartbeats or pulses of the acquired biological information,
wherein the reference information generating unit selects a desired extreme value from the extreme values of the amplitudes of the detected pieces of waveform information, and defines a time interval from a mid time between an extreme value time at which a previous extreme value is detected and the extreme value time of the selected extreme value up to a mid time between the extreme value time of the selected extreme value and an extreme value time at which an extreme value immediately after the selected extreme value is detected as a time interval corresponding to one heartbeat or one pulse, based upon the extreme value time at which the selected extreme value is detected, to cut out the waveform information of the biological information as the reference information.

15. The heartbeat measuring apparatus according to claim 13,
wherein the reference information generating unit includes a heartbeat extreme value detecting unit that detects an extreme value of an amplitude of the waveform information of heartbeats or pulses of the acquired biological information,
wherein the reference information generating unit selects a desired extreme value from the extreme values of the amplitudes of the detected heartbeat waveforms, calculates an extreme value time interval from the extreme value time at which the selected extreme value is detected to an extreme value time at which an extreme value immediately after the selected extreme value is detected, or an extreme value time interval from an extreme value time at which an extreme value immediately before the selected extreme value is detected to the extreme value time at which the selected extreme value is detected, and cuts out a portion of the waveform information of the biological information corresponding to a half or less of the calculated extreme value time interval from the extreme value time of the selected extreme value as the reference information.

16. The heartbeat measuring apparatus according to claim 13, wherein the reference information generating unit generates the reference information by cutting out information in a time interval corresponding to one heartbeat from the biological information acquired by the biological information acquiring unit.

17. The heartbeat measuring apparatus according to claim 13, wherein the reference information generating unit cuts out plural pieces of waveform information in time intervals, each corresponding to one heartbeat, from the biological information acquired by the biological information acquiring unit, to obtain an average of the cut-out plural pieces of waveform information as the reference information.

18. The heartbeat measuring apparatus according to claim 13, wherein the reference information generating unit cuts out plural pieces of waveform information in time intervals, each corresponding to one heartbeat, from the biological information acquired by the biological information acquiring unit, calculates waveform similarity between one of the cut-out pieces of waveform information and the other pieces of waveform information, and calculates an average of the pieces of waveform information other than the waveform information whose waveform similarity is lower than a predetermined waveform similarity value as the reference information.

19. The heartbeat measuring apparatus according to claim 13, wherein the reference information generating unit cuts out plural pieces of waveform information in time intervals, each corresponding to one heartbeat, from the biological information acquired by the biological information acquiring unit, compares each of the cut off pieces of waveform information with all the other pieces of waveform information to obtain a sum of the waveform similarities, and generates the waveform information with a highest sum of waveform similarities as the reference information.

20. A method of measuring a heartbeat comprising:
acquiring biological information derived from heartbeats, pulses or the like of a user;
detecting a body movement of the user;
generating reference information, which serves as a reference indicating the heartbeats or pulses for comparison, based upon the acquired biological information after completion of a detection of the body movement;
calculating a waveform similarity between the reference information and biological information acquired after the reference information is generated;
acquiring an extreme value time at which an extreme value is obtained by specifying the extreme value of the heartbeats or pulses from a plurality of waveform similarities; and
acquiring desired information on a living body of the user by analyzing the biological information of the user based upon a time interval between the extreme value times.
